(19) United States
(12) Patent Application Publication
(10) Pub. No.: US 2024/0407881 A1
(43) Pub. Date: Dec. 12, 2024

(54) OPTICAL STRUCTURE FOR SURGICAL MICROSCOPES AND SURGICAL MICROSCOPE

(71) Applicant: ZUMAX MEDICAL CO., LTD., Suzhou (CN)

(72) Inventors: Jilong Wang, Suzhou (CN); Jianyue Li, Suzhou (CN); Xiaoguang Yang, Suzhou (CN); Jiuqi Xu, Suzhou (CN); Tao Qiu, Suzhou (CN); Weizhong Zhou, Suzhou (CN)

(73) Assignee: ZUMAX MEDICAL CO., LTD., Suzhou, JS (CN)

(21) Appl. No.: 18/813,355

(22) Filed: Aug. 23, 2024

Related U.S. Application Data
(63) Continuation of application No. PCT/CN2023/131454, filed on Nov. 14, 2023.

(30) Foreign Application Priority Data
Nov. 15, 2022 (CN) 202211427023.1

(51) Int. Cl. A61B 90/20

(52) U.S. Cl. CPC A61B 90/20

(57) ABSTRACT

An optical structure is provided including a turning unit and a binocular unit which are sequentially arranged along an optical path direction. The turning unit includes a right-angle roof prism. The binocular unit includes a prism/lens group, a first right-angle prism, a second right-angle prism, and an ocular. The prism/lens group includes a small objective lens, or the first right-angle prism and the second right-angle prism are replaced by a rhombic prism. The optical structure is designed as “inverting-inverting”, and the right-angle roof prism shortens an optical length of an internal optical path of the turning unit by about half, which is more conducive to the reduction of the light vignetting of an optical path of observation. Two internal reflections prevent a mirroring phenomenon, and turn an image by 180 degrees, so that an inverting prism can be omitted from the binocular unit.

### CROSS-REFERENCE TO RELATED APPLICATIONS

This application is a continuation of International Application No. PCT/CN2023/131454, filed on Nov. 14, 2023, which claims priority to Chinese Application No. 202211427023.1, filed on Nov. 15, 2022, both of which are hereby incorporated by reference in their entireties.

### FIELD OF TECHNOLOGY

The following belongs to the technical field of medical apparatuses, and in particular, to an optical structure for surgical microscopes and a surgical microscope.

### BACKGROUND

The surgical microscope is a type of medical precision optical equipment, which is used for clinical microscopic observation and surgical treatment. As a core component, an optical microscopic observation system includes a main lens body and a binocular tube, and various accessories may also be arranged in a parallel optical path therein to achieve different additional functions.

In order to provide a comfortable ergonomic posture, an optical path turning structure is usually added over the main lens body of a conventional surgical microscope to turn the optical path by 90 degrees, so that the binocular tube can be horizontally arranged. Therefore, on the one hand, a line-of-sight height can be reduced and an operating space can be enlarged, and on the other hand, a horizontal observation distance can be increased, so that a doctor can be kept in a comfortable sitting position. Considering the need to avoid mirroring and keep an image erect, optical path turning structures in the conventional art are all implemented by pentaprisms. The Chinese patent publication No. CN211123465U discloses a surgical microscope, which adopts the combination of a pentaprism and a Schmidt prism or other prisms to implement the turning and splitting of light.

See a surgical microscope disclosed in the Chinese patent publication No. CN216148235U, it also uses a pentaprism as an optical path turning element, and the specific structure is shown in FIGS. 1 and 2.

However, due to the limitation of the structure of the pentaprism, an internal optical path is long, and an air gap of a fixing structure is large as well. As a result, the vignetting of an observation system is increased, and even the light in a marginal field of view is completely blocked, resulting in dark corners. Moreover, a combined optical path in a binocular tube and an ocular of a conventional surgical microscope is the same as that of a Kepler telescope. In order to observe an erect image, it is necessary to add an inverting prism into the optical path of the binocular tube. The most common Porro prism is generally composed of three right-angle prisms stuck together, as shown in FIGS. 17a, 17b, 17c and 17d. This prism structure is complex, both processing and assembly costs are high, and the problem of a long internal optical path still exists, which will make a small objective lens group complex, further increasing design and manufacturing costs.

### SUMMARY

An aspect relates to an optical structure for surgical microscopes in order to solve the problem of long internal optical paths in optical structures.

In order to achieve the aforementioned aspect, a first technical solution adopted by the present disclosure is as follows:

An optical structure for surgical microscopes includes a turning unit and a binocular unit, the turning unit and the binocular unit being sequentially arranged along an optical path direction, wherein:
the turning unit includes a right-angle roof prism; and
the binocular unit includes a prism/lens group, a first right-angle prism, a second right-angle prism, and an ocular, the prism/lens group, the first right-angle prism, the second right-angle prism and the ocular being sequentially arranged along the optical path direction, and the prism/lens group including a small objective lens.

According to the aforementioned technical solution, in some embodiments, an object plane beam is turned by 180 degrees through the right-angle roof prism, and continues to be turned by 180 degrees after passing through the prism/lens group, the first right-angle prism and the second right-angle prism, so that an erect real image is generated and observed by the ocular.

According to the aforementioned technical solution, in some embodiments, the prism/lens group is an angle-adjustable prism/lens group, which is applicable to a binocular tube with a rotatable connection component and arranged in the rotatable connection component, and can be adjusted by the rotation of the rotatable connection component.

In some embodiments, the angle-adjustable prism/lens group further includes a third right-angle prism, a fourth right-angle prism, and a fifth right-angle prism, the third right-angle prism, the small objective lens, the fourth right-angle prism and the fifth right-angle prism being sequentially arranged along the optical path direction.

In some embodiments, the third right-angle prism and the fifth right-angle prism can be rotated around an optical axis relative to the fourth right-angle prism, with an angle of rotation being always constant.

According to the aforementioned technical solution, in some embodiments, the prism/lens group includes a half-pentaprism, the small objective lens and the half-pentaprism being sequentially arranged along the optical path direction, wherein the half-pentaprism is used as an optical structure of a binocular tube adopting a 45° inclined binocular tube, and compared with a conventional Schmidt roof prism, the half-pentaprism adopted is relatively simple in processing, assembly and correction and low in cost.

According to the aforementioned technical solution, in some embodiments, the prism/lens group does not include a meniscus lens.

According to the aforementioned technical solution, in some embodiments, the small objective lens includes a first cemented doublet group with a positive focal power, and meets:

50 mm<|f_{G1}|<200 mm,

|R₁|/φ_{1/2}<15
wherein f_{G1} is a focal length of the first cemented doublet group,
R₁ is a curvature radius of the cemented surface of the cemented doublet group, and
φ₁ is an effective aperture of the cemented surface.

In some embodiments, the ocular includes a second cemented doublet group and a single lens which are sequentially arranged along the optical path direction, and meets:

0.5<f_{L3}/f_{G1}=2
wherein f_{L3} is a focal length of the second cemented doublet group.

According to the aforementioned technical solution, in some embodiments, the binocular unit further includes a field lens, which is a single lens.

According to the aforementioned technical solution, in some embodiments, the binocular unit further includes a diaphragm.

According to the aforementioned technical solution, in some embodiments, the binocular unit further includes a field lens and a diaphragm, the small objective lens, the first right-angle prism, the second right-angle prism, the field lens, the diaphragm and the ocular being sequentially arranged along the optical path direction; the binocular unit is applicable to a binocular tube adopting a straight binocular tube; the two right-angle prisms are easier to assemble and adjust, a distance in between is adjustable, a pupil distance range is convenient to adjust, the design and assembly of a mechanical structure are facilitated, the occupied space is small, the weight is light, processing, assembly and correction are convenient, and the cost is low.

According to the aforementioned technical solution, in some embodiments, the prism/lens group further includes a third right-angle prism, a fourth right-angle prism, and a fifth right-angle prism; the binocular unit further includes a field lens and a diaphragm, the third right-angle prism, the small objective lens, the fourth right-angle prism, the fifth right-angle prism, the first right-angle prism, the second right-angle prism, the field lens, the diaphragm and the ocular being sequentially arranged along the optical path direction; the binocular unit is applicable to a binocular tube with a rotatable connection component, the two right-angle prisms are easier to assemble and adjust, a distance in between is adjustable, a pupil distance range is convenient to adjust, and an aperture of the first right-angle prism can be reduced according to an optical path, facilitating the design and assembly of a mechanical structure; and moreover, because an optical length after the small objective lens is greatly reduced, there is no need to additionally add a thick meniscus lens to adjust the position of an image plane, so that the system is simplified and the cost is reduced.

According to the aforementioned technical solution, in some embodiments, the prism/lens group further includes a half-pentaprism, the small objective lens, the half-pentaprism, the first right-angle prism, the field lens, the second right-angle prism, the diaphragm and the ocular being sequentially arranged along the optical path direction; the binocular unit is applicable to a binocular tube adopting a 45° inclined binocular tube, processing, assembly and correction are relatively simple, and the cost is low.

In order to achieve the aforementioned aspect, a second technical solution adopted by the present disclosure is as follows:

An optical mechanism for surgical microscopes includes a turning unit and a binocular unit which are sequentially arranged along an optical path direction, wherein
the turning unit includes a right-angle roof prism; and
the binocular unit includes a prism/lens group, a rhombic prism and an ocular which are sequentially arranged along an optical path direction, wherein the prism/lens group includes a small objective lens.

According to the aforementioned technical solution, in some embodiments, an object plane beam is turned by 180 degrees through the right-angle roof prism, and continues to be turned by 180 degrees after passing through the prism/lens group and the rhombic prism, so that an erect real image is generated and observed by the ocular.

According to the aforementioned technical solution, in some embodiments, the prism/lens group further includes a third right-angle prism, a fourth right-angle prism, and a fifth right-angle prism; the binocular unit further includes a field lens and a diaphragm, the third right-angle prism, the small objective lens, the fourth right-angle prism, the fifth right-angle prism, the rhombic prism, the field lens, the diaphragm and the ocular being sequentially arranged along the optical path direction; the binocular unit is applicable to a binocular tube with a rotatable connection component; the rhombic prism is adopted, the left prism and the right prism are identical, processing, assembly and correction are relatively simple, and the cost is low.

Another aspect of the present disclosure is to provide a surgical microscope.

In order to achieve the aforementioned aspect, a technical solution adopted by the present disclosure is as follows:

A surgical microscope includes a microscope body, a turning extender, and a binocular tube, the turning extender being connected to the microscope body and the binocular tube being connected to the turning extender, and the surgical microscope further includes the aforementioned optical structure, the turning unit being arranged in the turning extender and the binocular unit being arranged in the binocular tube.

According to the aforementioned technical solution, in some embodiments, the binocular tube is provided with a rotatable connection component which is connected with the turning extender and can be rotated for adjustment in a vertical direction relative to the turning extender, and the prism/lens group is an angle-adjustable prism/lens group, which is arranged in the rotatable connection component.

According to the aforementioned technical solution, in some embodiments, the binocular tube is a straight binocular tube.

According to the aforementioned technical solution, in some embodiments, the binocular tube is a 45° inclined binocular tube.

According to the aforementioned technical solution, in some embodiments, a fixed seat is arranged in the turning extender, and the right-angle roof prism is arranged on the fixed seat.

According to the aforementioned technical solution, in some embodiments, the binocular tube and the turning extender are detachably connected with each other.

Due to the application of the aforementioned technical solutions, the present disclosure has the following advantages in comparison with the conventional art:
1. the optical structure according to the present disclosure reduces the optical length of an internal optical path of the turning extender by about half, which is more conductive to the reduction of the light vignetting of an optical path of observation;
2. the optical structure according to the present disclosure avoids a mirroring phenomenon through two internal reflections of the right-angle roof prism, and turns the image by 180 degrees, so a complex and high-cost inverting prism can be omitted from the binocular unit;
3. the optical structure according to the present disclosure avoids an excessive optical length caused by the necessary arrangement of an inverting prism in a binocular tube in the conventional art, decreasing the difficulty of the design of the small objective lens and simplifying the structure of the optical lens group;
4. the optical structure according to the present disclosure improves the observation effect of the optical system, simplifies the optical structure of the lens group and the prism group, decreases the difficulty of assembly and correction, and reduces production and manufacturing costs.

### BRIEF DESCRIPTION OF THE DRAWINGS

FIG. 1 is a schematic front view of a surgical microscope in the conventional art;

FIG. 2 is a schematic top view of the surgical microscope in the conventional art;

FIG. 3a is a schematic diagram of an optical path of an optical structure in the conventional art (a binocular tube with a rotatable connection component);

FIG. 3b is a schematic diagram of an optical path of an optical structure in the conventional art (a binocular tube adopting a straight binocular tube);

FIG. 4a is a schematic front view of a surgical microscope in Embodiment 1;

FIG. 4b is a schematic top view of the surgical microscope in Embodiment 1;

FIG. 4c is a schematic bottom view of the surgical microscope in Embodiment 1;

FIG. 5 is a schematic diagram of an optical path of an optical structure in Embodiment 1;

FIG. 6 is a schematic diagram of an optical path of a binocular unit in the optical structure in Embodiment 1;

FIG. 7 is a schematic diagram of an optical path of an optical structure in Embodiment 2;

FIG. 8 is a schematic diagram of an optical path of a binocular unit in the optical structure in Embodiment 2;

FIG. 9 is a schematic diagram of a straight binocular tube in Embodiment 3;

FIG. 10 is a schematic diagram of an optical path of a binocular unit in an optical structure in Embodiment 3;

FIG. 11 is a schematic diagram of a 45° inclined binocular tube in Embodiment 4;

FIG. 12 is a schematic diagram of an optical path of a binocular unit in an optical structure in fourth Embodiment 4;

FIG. 13 is a schematic diagram of an optical path of a binocular unit in an optical structure in a comparative example;

FIG. 14 is a schematic diagram of a pentaprism;

FIG. 15a schematically depicts diagrams of various right-angle roof prisms;

FIG. 15b schematically depicts diagrams of various right-angle roof prisms;

FIG. 16 is a schematic diagram of superposition of a pentaprism and a right-angle roof prism;

FIG. 17a schematically depicts diagrams of various Porro prisms;

FIG. 17b schematically depicts diagrams of various Porro prisms;

FIG. 17c schematically depicts diagrams of various Porro prisms;

FIG. 17d schematically depicts diagrams of various Porro prisms;

FIG. 18a schematically depicts diagrams of various rhombic prisms; and

FIG. 18b schematically depicts diagrams of various rhombic prisms.

### DETAILED DESCRIPTION OF THE EMBODIMENTS

The technical solutions of the present disclosure will be clearly and fully described below with reference to the accompanying drawings, and it is obvious that the described embodiments are part of embodiments of the present disclosure rather than all of them. Based on the examples of the present disclosure, all other examples obtained by those of ordinary skilled in the art without creative efforts shall fall within the protection scope of the present disclosure.

In the description of the present disclosure, It should be noted that directions or positional relations indicated by terms, such as “central”, “upper”, “lower”, “left”, “right”, “vertical”, “horizontal”, “internal”, “external”, etc., are directions or positional relations shown in the accompanying drawings, which are merely intended to conveniently describe the present disclosure and simplify description rather than indicate or imply that the indicated device or elements must have specific directions and be structured and operated according to the specific directions, and therefore should not be interpreted as limitations on the present disclosure. In addition, the terms “first”, “second” and “second” are merely intended for description rather than understood as indicating or implying relative importance.

### Embodiment 1

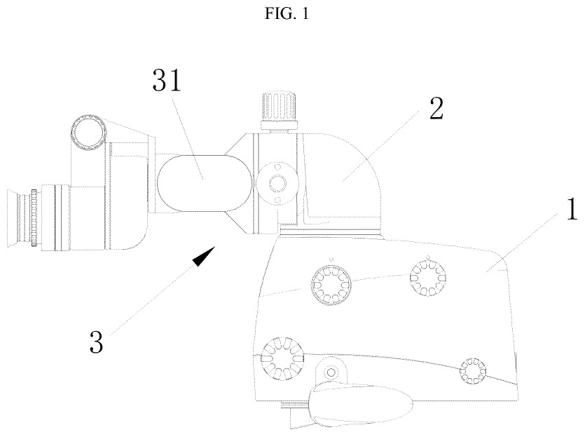

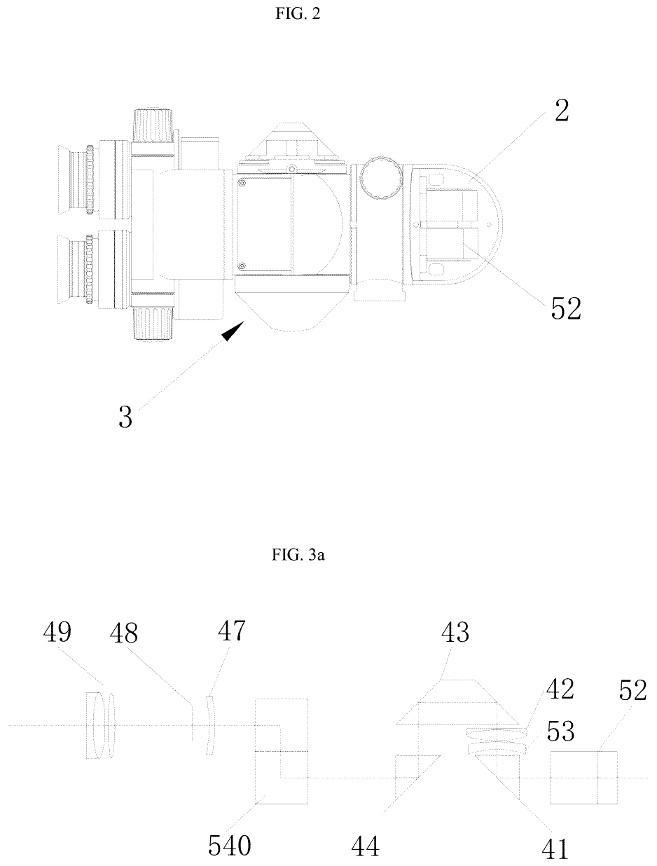

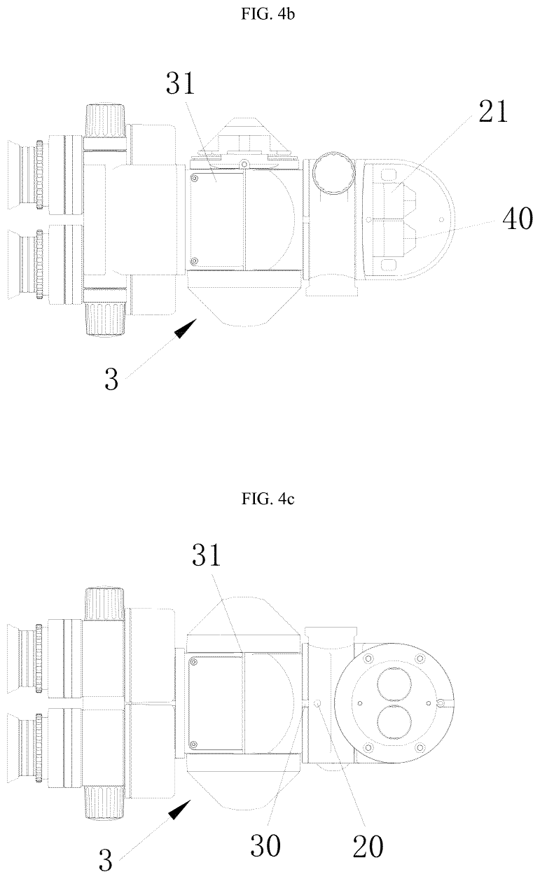

As shown in FIG. 1, a surgical microscope includes a microscope body 1, a turning extender 2, and a binocular tube 3, where the turning extender 2 is connected to the microscope body 1, and the binocular tube 3 is connected to the turning extender 2. As shown in FIG. 4c, the binocular tube 3 and the turning extender 2 may be detachably connected with each other through, for example, a positioning hole 30 and a locking hole 20. The binocular tube 3 is provided with a rotatable connection component 31, and the binocular tube 3 can be rotated for adjustment in a vertical direction relative to the turning extender 2 by the rotatable connection component 31. As a common structure of the binocular tube, the rotatable connection component 31 is not related to the inventive points of the present application, and therefore will not be repeated herein.

A corresponding optical structure is arranged in the surgical microscope. The optical structure includes a turning unit and a binocular unit which are sequentially arranged along an optical path direction, the turning unit is arranged in the turning extender 2, and the binocular unit is arranged in the binocular tube 3.

As shown in FIGS. 4a and 4b, the turning unit includes a right-angle roof prism 40, a fixed seat 21 is arranged in the turning extender 2, and the right-angle roof prism 40 is arranged on the fixed seat 21, that is, the right-angle roof prism 40 is used as an optical path turning element in the present embodiment.

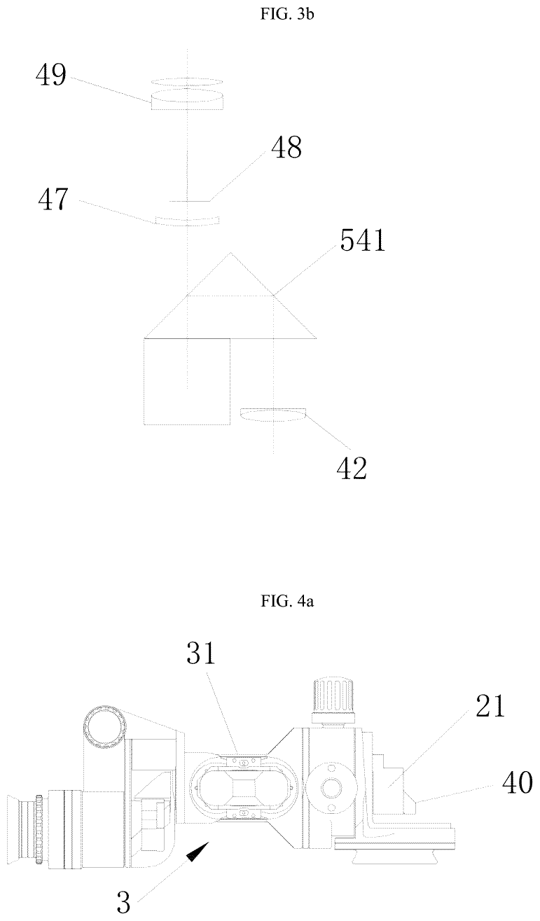

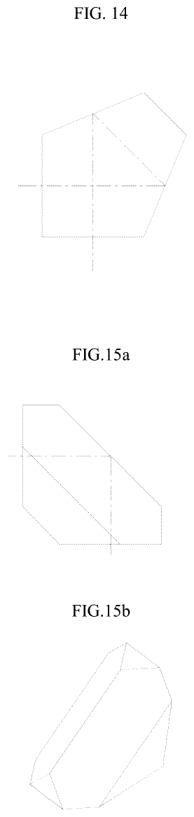

As shown in FIG. 3a, a turning unit in a conventional optical structure adopts a pentaprism 52. It has been discovered from the comparison between both that an optical length of the pentaprism 52 (as shown in FIG. 14) is 3.41D₀ (D₀ is a clear aperture of the prism), an optical length of the right-angle roof prism 40 (as shown in FIGS. 15a and 15b) is 1.73D₀, and the optical length of the right-angle roof prism 40 is 1.68D₀ shorter than that of the pentaprism 52. Calculated according to a minimum clear aperture of 18 mm, an optical path of the right-angle roof prism 40 is 30.24 mm shorter than that of the pentaprism 52 (an equivalent air gap is about 20 mm).

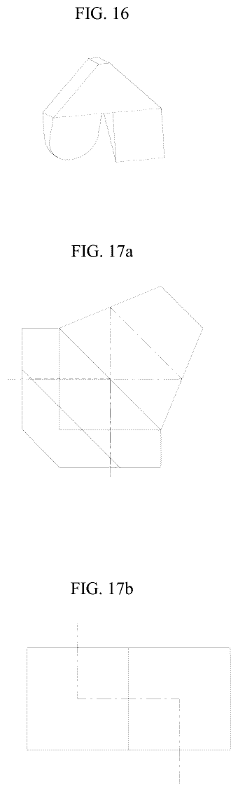

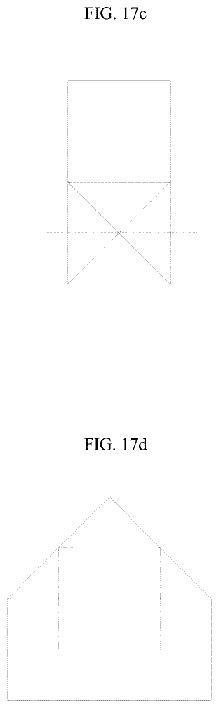

A turning point of an optical axis of the right-angle roof prism 40 adopted in the present application is higher than a midpoint. When the optical axis is raised by the same distance, its edge is closer to a fixed surface than the pentaprism 52 (as shown in FIG. 16), which further reduces the air gap. Calculated according to the minimum clear aperture of 18 mm, the air gap of 13.2 mm is replaced by glass, so the air gap is reduced by about 4.5 mm.

Therefore, the use of the right-angle roof prism 40 in this parallel optical path significantly shortens the optical path, so that a projection height of main light in a marginal field of view is greatly reduced, effectively reducing edge vignetting and avoiding the phenomenon of the edge of an image being dim or even blocked.

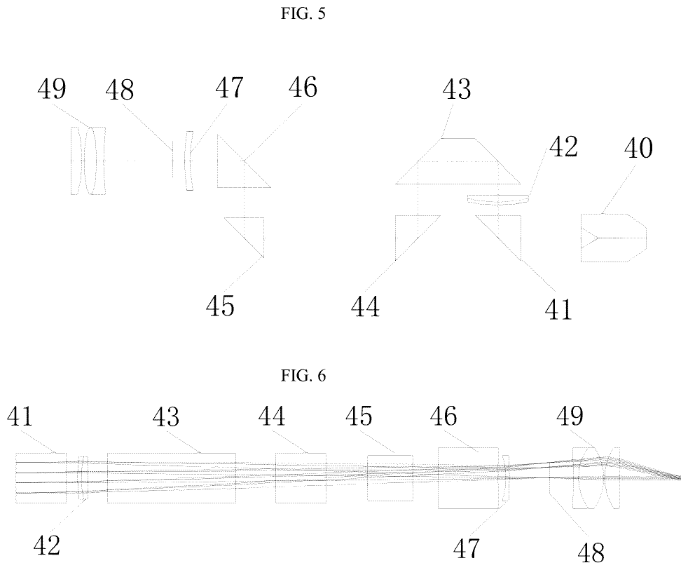

As shown in FIG. 6, the binocular unit includes a prism/lens group, a first right-angle prism 45, a second right-angle prism 46, a field lens 47, a diaphragm 48 and an ocular 49 which are sequentially arranged along the optical path direction. The prism/lens group is an angle-adjustable prism/lens group, which is arranged in the rotatable connection component 31. In the present embodiment, the angle-adjustable prism/lens group includes a third right-angle prism 41, a small objective lens 42, a fourth right-angle prism 43, and a fifth right-angle prism 44.

As shown in FIG. 5, the whole optical structure includes a right-angle roof prism 40, a third right-angle prism 41, a small objective lens 42, a fourth right-angle prism 43, a fifth right-angle prism 44, a first right-angle prism 45, a second right-angle prism 46, a field lens 47, a diaphragm 48 and an ocular 49 which are sequentially arranged along the optical path direction, and an object plane beam is turned by 180 degrees through the right-angle roof prism 40, and continues to be turned by 180 degrees after passing through the third right-angle prism 41, the small objective lens 42, the fourth right-angle prism 43, the fifth right-angle prism 44, the first right-angle prism 45, the second right-angle prism 46 and the field lens 47, so that an erect real image is generated at the diaphragm 48 and observed by the ocular 49.

As shown in FIG. 3a, a binocular unit of the optical structure in the conventional art adopts a Porro prism 540. It has been discovered in comparison with the first right-angle prism 45 and the second right-angle prism 46 in the present application that the Porro prism 540 is formed by three prisms which are stuck together, and it is necessary to distinguish left and right combination methods, so processing, assembly and correction are relatively complex and the cost is high. Moreover, a focal length of a small objective lens 42 of a conventional binocular tube is generally 170 mm. Due to the requirement of a subsequent optical path structure (particularly, an excessive optical length of the Porro prism 540), it is necessary to add a thick meniscus lens 53 to move an optical principal plane of the small objective lens 42 backward. However, the thick meniscus lens 53 is difficult to center and highly sensitive to a curvature radius, having a strict requirement for a tolerance, so both processing and manufacturing costs are high.

Since two right-angle prisms (the first right-angle prism 45 and the second right-angle prism 46) are adopted in the present application, assembly and adjustment are easier, a distance in between is adjustable, a pupil distance range is convenient to adjust, and according to an optical path, the aperture of the first right-angle prism 45 may be reduced, facilitating the design and assembly of a mechanical structure (a distance between the centers of two optical paths is generally only 22 mm). Moreover, because an optical length after the small objective lens 42 is greatly reduced, there is no need to additionally add a thick meniscus lens 53 to adjust the position of an image plane, so that the system is simplified and the cost is reduced.

In the present embodiment, the small objective lens 42 is a first cemented doublet group (two lenses) with a positive focal power, and meets:

50 mm<|f_{G1}|<200 mm

$$\frac{\vert {R}_{2} \vert}{{φ}_{1 / 2}} < 15 ,$$

where f_{G1} is a focal length of the first cemented doublet group, R₁ is a curvature radius of the cemented surface of the cemented doublet group, and φ1 is an effective aperture of the cemented surface.

The field lens 47 is a single lens with a negative focal power.

The ocular 49 includes a single lens and a second cemented doublet group (two lenses) which are sequentially arranged along the optical path direction, and meets:

$$0.5 < {f}_{L 3} / {f}_{G 1} < 2 ,$$

where f_{L3} is a focal length of the second cemented doublet group.

As shown in FIG. 6 and Table 1, optical parameters of the binocular unit in the present embodiment are provided:

| Plane | Radius | Thickness | Nd | Vd | Semiaperture |
|---|---|---|---|---|---|
| 1 | Infinity | 20 | 1.52 | 64.2 | 10 |
| 2 | Infinity | 4.5 | | | 10 |
| 3 | 61.85 | 1.5 | 1.59 | 29.9 | 8.5 |
| 4 | 37.26 | 2.5 | 1.49 | 57.4 | 8.5 |
| 5 | Infinity | 8 | | | 8.5 |
| 6 | Infinity | 51 | 1.52 | 64.2 | 10 |
| 7 | Infinity | 16 | | | 10 |
| 8 | Infinity | 20 | 1.52 | 64.2 | 10 |
| 9 | Infinity | 16.6 | | | 10 |
| 10 | Infinity | 18 | 1.52 | 64.2 | 9 |
| 11 | Infinity | 10 | | | 9 |
| 12 | Infinity | 24 | 1.52 | 64.2 | 12 |
| 13 | Infinity | 3 | | | 12 |
| 14 | −30.72 | 1.5 | 1.49 | 57.4 | 9 |
| 15 | −98.9 | 16 | | | 9 |
| 16 | Infinity | 9.5 | | | |
| 17 | −238.9 | 2 | 1.85 | 23.8 | 12 |
| 18 | 22.839 | 10 | 1.62 | 60.3 | 12 |
| 19 | −17 | 0.5 | | | 12 |
| 20 | 28.18 | 6 | 1.62 | 60.3 | 12 |
| 21 | Infinity | 23.5 | | | 12 |

where the radius is a curvature radius of the lens surface, the thickness is a thickness of the lens center, and Nd is a refractive index of d light (wavelength: 589.3 nm) in optical glass; and Vd is an abbe number.

The optical structure adopted in the present embodiment is designed as “inverting-inverting”. Compared with the conventional optical structure in FIG. 3a, its internal optical path is shortened by about half, which is more conducive to the reduction of the light vignetting of an optical path of observation. Two internal reflections prevent a mirroring phenomenon, and turn an image by 180 degrees, so that an inverting prism can be omitted from the subsequent binocular unit, so the structure is simple and the cost is low.

### Embodiment 2

The present embodiment is substantially the same as the first embodiment, except that in the present embodiment, a rhombic prism 50 is adopted in the binocular unit to replace the first right-angle prism 45 and the second right-angle prism 46 in Embodiment 1.

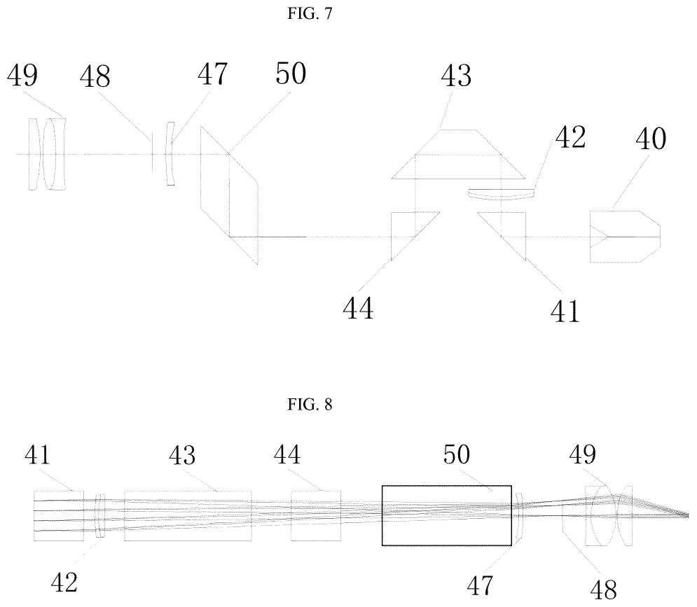

As shown in FIGS. 7 and 8, the whole optical structure includes a right-angle roof prism 40, a third right-angle prism 41, a small objective lens 42, a fourth right-angle prism 43, a fifth right-angle prism 44, an rhombic prism 50, a field lens 47, a diaphragm 48 and an ocular 49 which are sequentially arranged along an optical path direction, and an object plane beam is turned by 180 degrees through the right-angle roof prism 40, and continues to be turned by 180 degrees after passing through the third right-angle prism 41, the small objective lens 42, the fourth right-angle prism 43, the fifth right-angle prism 44 and the rhombic prism 50, so that an erect real image is generated, which is then observed by the ocular 49 through the field lens 47 and the diaphragm 48.

As shown in FIG. 3a, the binocular unit of the optical structure in the conventional art adopts a Porro prism 540. It has been discovered in comparison with the rhombic prism 50 in the present application that the optical length of the Porro prism 540 is 4D₀, and the optical length of the rhombic prism 50 is 2D₀. The optical length of the rhombic prism 50 is 2D₀ shorter than that of the Porro prism 540, and calculated according to a minimum clear aperture of 22 mm, the optical length of the rhombic prism 50 is 44 mm shorter than that of the Porro prism 540.

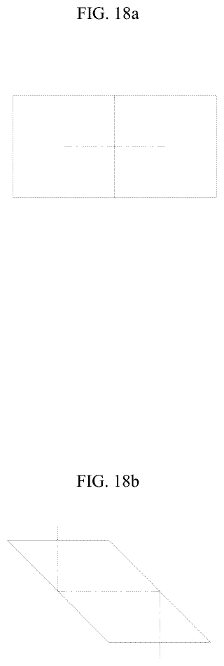

As shown in FIGS. 18a and 18b, the left and right prisms of the rhombic prism 50 adopted in the present application are identical, so processing, assembly and correction are relatively complex and the cost is high.

As shown in FIG. 8 and Table 2, optical parameters of the binocular unit in the present embodiment are provided:

| Plane No. | Radius | Thickness | Nd | Vd | Semiaperture |
|---|---|---|---|---|---|
| 1 | Infinity | 20 | 1.52 | 64.2 | 10 |
| 2 | Infinity | 4.5 | | | 10 |
| 3 | 61.85 | 1.5 | 1.59 | 29.9 | 8.5 |
| 4 | 37.26 | 2.5 | 1.49 | 57.4 | 8.5 |
| 5 | Infinity | 8 | | | 8.5 |
| 6 | Infinity | 51 | 1.52 | 64.2 | 10 |
| 7 | Infinity | 16 | | | 10 |
| 8 | Infinity | 20 | 1.52 | 64.2 | 10 |
| 9 | Infinity | 16.6 | | | 10 |
| 10 | Infinity | 52 | 1.52 | 64.2 | 9 |
| 11 | Infinity | 3 | | | 12 |
| 12 | −30.72 | 1.5 | 1.49 | 57.4 | 9 |
| 13 | −98.9 | 18 | | | 9 |
| 14 | Infinity | 9.5 | | | |
| 15 | −238.9 | 2 | 1.85 | 23.8 | 12 |
| 16 | 22.839 | 10 | 1.62 | 60.3 | 12 |
| 17 | −17 | 0.5 | | | 12 |
| 18 | 28.18 | 6 | 1.62 | 60.3 | 12 |
| 19 | Infinity | 23.5 | | | 12 |

### Embodiment 3

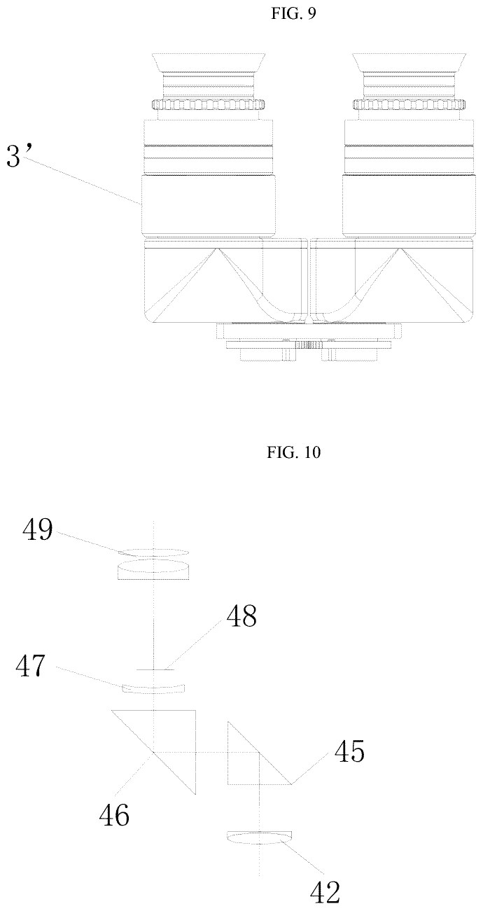

The present embodiment is substantially the same as Embodiment 1, except that in the present embodiment, the binocular tube is a straight binocular tube 3′, as shown in FIG. 9. The straight binocular tube 3′ is not provided with a rotatable connection component 31, and an optical axis of an ocular 49 is kept parallel to that of a small objective lens 42. The straight binocular tube 3′ is directly connected with a turning extender 2, but the straight binocular tube 3′ cannot be rotated for adjustment in a vertical direction relative to the turning extender 2. At this point, a prism/lens group only includes the small objective lens 42.

As shown in FIG. 10, a binocular unit includes the small objective lens 42, a first right-angle prism 45, a second right-angle prism 46, a field lens 47, a diaphragm 48 and the ocular 49 which are sequentially arranged along an optical path direction.

As shown in FIG. 3b, the binocular unit of the optical structure in the conventional art adopts a Porro prism 541. It has been discovered in comparison with the first right-angle prism 45 and the second right-angle prism 46 in the present application that the Porro prism 540 is formed by two large right-angle prisms which are stuck together, and it is necessary to distinguish left and right combination methods. As a result, a large structural space is occupied, the weight is heavy, processing, assembly and correction are relatively complex, and the cost is high.

Since two right-angle prisms (the first right-angle prism 45 and the second right-angle prism 46) are adopted in the present application, assembly and adjustment are easier, a distance in between is adjustable, a pupil distance range is convenient to adjust, the design and assembly of a mechanical structure is facilitated, the occupied space is small, the weight is light, processing, assembly and correction are convenient, and the cost is low.

### Embodiment 4

The present embodiment is substantially the same as Embodiment 1, except that in the present embodiment, the binocular tube is a 45° inclined binocular tube 3″, as shown in FIG. 11. The inclined binocular tube 3″ is not provided with a rotatable connection component 31, and an optical axis of an ocular 49 keeps an included angle of 45° with an optical axis of a small objective lens 42. The inclined binocular tube 3″ is directly connected with a turning extender 2, but the inclined binocular tube 3″ cannot be rotated for adjustment in a vertical direction relative to the turning extender 2. At this point, a prism/lens group includes the small objective lens 42 and a half-pentaprism 51 which are sequentially arranged along an optical path direction.

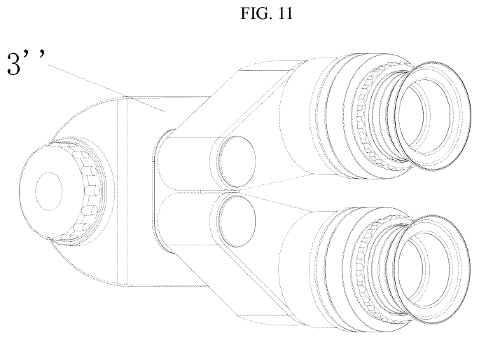

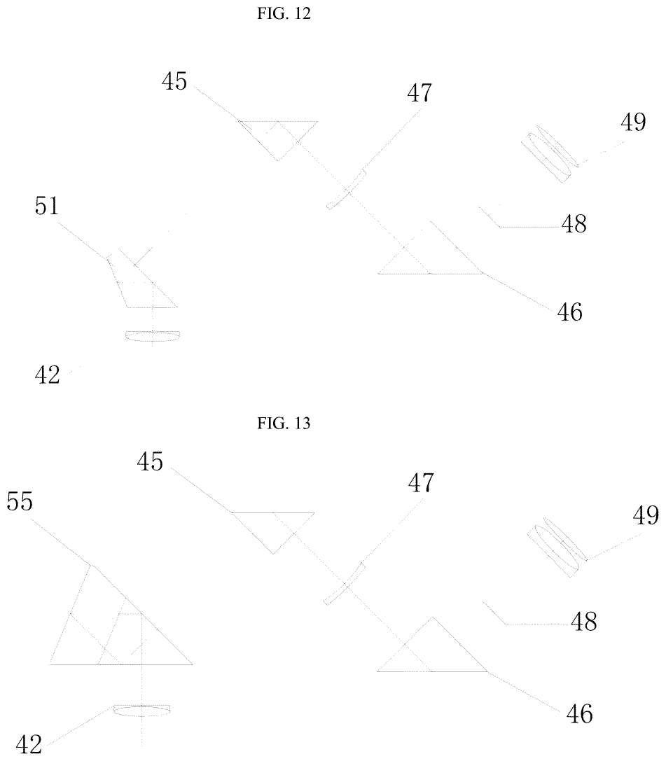

As shown in FIG. 12, a binocular unit includes the small objective lens 42, the half-pentaprism 51, a first right-angle prism 45, a field lens 47, a second right-angle prism 46, a diaphragm 48 and the ocular 49 which are sequentially arranged along an optical path direction.

Compared with the Schmidt roof prism 55 adopted in the optical structure shown in FIG. 13, the half-pentaprism 51 adopted in the present embodiment is relatively simple to process, assemble and correct and low in cost.

The aforementioned embodiments are merely intended to describe the technical concept and characteristics of the present disclosure. Their purpose is to enable those familiar with this technique to comprehend and implement the content of the present disclosure, and the protection scope of the present disclosure cannot be limited hereby. Any equivalent alteration or modification which is made according to the spirit of the present disclosure shall be covered by the protection scope of the present disclosure.

What is claimed is:

1. An optical structure for surgical microscopes, comprising a turning unit and a binocular unit, the turning unit and the binocular unit being sequentially arranged along an optical path direction, wherein
the turning unit comprises a right-angle roof prism; and
the binocular unit comprises a prism/lens group, a first right-angle prism, a second right-angle prism, and an ocular, the prism/lens group, the first right-angle prism, the second right-angle prism and the ocular being sequentially arranged along the optical path direction, and the prism/lens group comprising a small objective lens.

2. The optical structure for surgical microscopes according to claim 1, wherein an object plane beam is turned by 180 degrees through the right-angle roof prism, and continues to be turned by 180 degrees after passing through the prism/lens group, the first right-angle prism and the second right-angle prism, so that an erect real image is generated and observed by the ocular.

3. The optical structure for surgical microscopes according to claim 1, wherein the prism/lens group is an angle-adjustable prism/lens group.

4. The optical structure for surgical microscopes according to claim 3, wherein the angle-adjustable prism/lens group further comprises a third right-angle prism, a fourth right-angle prism, and a fifth right-angle prism, the third right-angle prism, the small objective lens, the fourth right-angle prism and the fifth right-angle prism being sequentially arranged along the optical path direction.

5. The optical structure for surgical microscopes according to claim 4, wherein the third right-angle prism and the fifth right-angle prism can be rotated around an optical axis relative to the fourth right-angle prism, with an angle of rotation being always constant.

6. The optical structure for surgical microscopes according to claim 1, wherein the prism/lens group comprises a half-pentaprism, the small objective lens and the half-pentaprism being sequentially arranged along the optical path direction.

7. The optical structure for surgical microscopes according to claim 1, wherein the prism/lens group does not comprise a meniscus lens.

8. The optical structure for surgical microscopes according to claim 1, wherein the small objective lens comprises a first cemented doublet group with a positive focal power, and meets:
$$50 mm < \vert {f}_{G 1} \vert < 200 mm ,$$
$$\frac{\vert {R}_{1} \vert}{{φ}_{1 / 2}} < 15 ,$$
wherein f_{G1} is a focal length of the first cemented doublet group,
R₁ is a curvature radius of the cemented surface of the cemented doublet group, and
φ₁ is an effective aperture of the cemented surface.

9. The optical structure for surgical microscopes according to claim 8, wherein the ocular comprises a second cemented doublet group and a single lens which are sequentially arranged along the optical path direction, and meets:
$$0.5 < {f}_{L 3} / {f}_{G 1} < 2 ,$$
wherein f_{L3} is a focal length of the second cemented doublet group.

10. The optical structure for surgical microscopes according to claim 1, wherein the binocular unit further comprises a field lens and a diaphragm, the small objective lens, the first right-angled prism, the second right-angled prism, the field lens, the diaphragm and the ocular being sequentially arranged along the optical path direction.

11. The optical structure for surgical microscopes according to claim 1, wherein the prism/lens group further comprises a third right-angle prism, a fourth right-angle prism, and a fifth right-angle prism, and the binocular unit further comprises a field lens and a diaphragm, the third right-angle prism, the small objective lens, the fourth right-angle prism, the fifth right-angle prism, the first right-angle prism, the second right-angle prism, the field lens, the diaphragm and the ocular being sequentially arranged along the optical path direction.

12. The optical structure for surgical microscopes according to claim 1, wherein the prism/lens group further comprises a half-pentaprism, the small objective lens, the half-pentaprism, the first right-angle prism, the field lens, the second right-angle prism, the diaphragm and the ocular being sequentially arranged along the optical path direction.

13. An optical mechanism for surgical microscopes, comprising a turning unit and a binocular unit which are sequentially arranged along an optical path direction, wherein
the turning unit comprises a right-angle roof prism; and
the binocular unit comprises a prism/lens group, a rhombic prism and an ocular which are sequentially arranged along an optical path direction, wherein the prism/lens group comprises a small objective lens.

14. The optical structure for surgical microscopes according to claim 13, wherein an object plane beam is turned by 180 degrees through the right-angle roof prism, and continues to be turned by 180 degrees after passing through the prism/lens group and the rhombic prism, so that an erect real image is generated and observed by the ocular.

15. The optical structure for surgical microscopes according to claim 13, wherein the prism/lens group further comprises a third right-angle prism, a fourth right-angle prism, and a fifth right-angle prism; the binocular unit further comprises a field lens and a diaphragm, the third right-angle prism, the small objective lens, the fourth right-angle prism, the fifth right-angle prism, the rhombic prism, the field lens, the diaphragm and the ocular being sequentially arranged along the optical path direction.

16. A surgical microscope, comprising a microscope body, a turning extender, and a binocular tube, the turning extender being connected to the microscope body, and the binocular tube being connected to the turning extender, wherein the surgical microscope further comprises the optical structure according to claim 1, the turning unit is arranged in the turning extender, and the binocular unit is arranged in the binocular tube.

17. The surgical microscope according to claim 16, wherein the binocular tube is provided with a rotatable connection component which is connected with the turning extender and can be rotated for adjustment in a vertical direction relative to the turning extender, and the prism/lens group is an angle-adjustable prism/lens group, which is arranged in the rotatable connection component.

18. The surgical microscope according to claim 16, wherein the binocular tube is a straight binocular tube, or the binocular tube is a 45° included binocular tube.

19. The surgical microscope according to claim 16, wherein a fixed seat is arranged in the turning extender, and the right-angle roof prism is arranged on the fixed seat.

20. The surgical microscope according to claim 16, wherein the binocular tube and the turning extender are detachably connected with each other.
